(19) United States
(12) Patent Application Publication
(10) Pub. No.: US 2015/0167039 A1
(43) Pub. Date: Jun. 18, 2015

(54) METHOD FOR CHITOSAN PRODUCTION

(71) Applicant: Mycodev Group Inc., Fredericton (CA)

(72) Inventor: David Brown, Island View (CA)

(73) Assignee: MYCODEV GROUP INC., Fredericton (CA)

(21) Appl. No.: 14/568,275

(22) Filed: Dec. 12, 2014

Related U.S. Application Data
(60) Provisional application No. 61/915,845, filed on Dec. 13, 2013.

(51) Int. Cl. C12P 19/26

(52) U.S. Cl. CPC C12P 19/26

(57) ABSTRACT

The present disclosure relates to methods for chitosan production. In particular, the present disclosure relates to a method for chitosan production comprising growing a biomass comprising an organic acid-producing, chitosan-containing fungus in an aqueous medium. A chitosan-reduced biomass and a chitosan-enriched aqueous medium are obtained. The chitosan-reduced biomass is separated from the chitosan-enriched aqueous medium. The chitosan is then separated from the chitosan-enriched aqueous medium, for example by adding a base to the chitosan-enriched aqueous medium under conditions to precipitate the chitosan and separating the precipitated chitosan from the aqueous medium.

### CROSS-REFERENCE TO RELATED APPLICATIONS

The present application claims the benefit of priority from co-pending U.S. provisional application No. 61/915,845 filed on Dec. 13, 2013, the contents of which are incorporated herein by reference in their entirety.

### FIELD

The present disclosure relates to methods for chitosan production. In particular, the present disclosure relates to a method for chitosan production using an organic acid-producing, chitosan-containing fungus.

### BACKGROUND

Chitin and chitosan are members of the glycosaminoglycan family of polymers. Chitin is a linear polymer made up of N-acetyl-(D)-glucosamine (1) and optionally (D)-glucosamine (2) units which have been connected through β-(1,4) bonds (Scheme 1). Chitosan is a copolymer of N-acetyl-(D)-glucosamine and (D)-glucosamine units which have been connected through β-(1,4) bonds and is sometimes considered to be an N-deacetylated derivative of chitin. While the figure may vary, it is generally accepted in the literature that a polymer having 60% or greater (D)-glucosamine units is chitosan. For example, Aranaz et al. define chitosan as having 60% deacetylation i.e. 60% of the acetyl groups removed compared to a 100% acetylated chitin polymer.¹

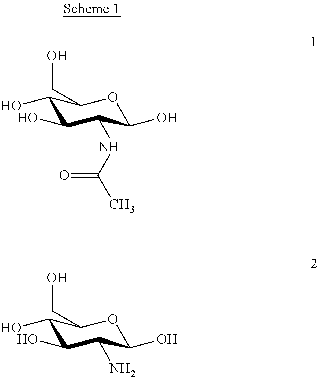

Chitin is found in nature in such large quantities that it is the second most abundant biopolymer after cellulose. For example, it is a component of crustaceans' exoskeletons such as crab and shrimp shells, fungal and algal cell walls as well as in the cuticle of insects. Today, the vast majority of chitin is extracted from crustacean sources; typically crab, crawfish and shrimp shells left over from food processors. Chitosan may be commercially produced from chitin via a process which deacetylates the chitin.

Known methods of producing chitosan from a crustacean source are typically costly from an environmental standpoint as they may be water intensive, require the use of large volumes of acids and base, and/or use a large amount of energy. Standard methods for making chitosan from crustacean waste typically comprise steps which include subjecting the crustacean shell waste to deproteinization, demineralization and deacetylation. If decolorized chitosan is desired, the process can include decolorizing the chitin prior to deacetylation. The process can also include other steps such as washing and/or drying steps.

Deproteinization typically comprises treatment of ground crustacean shell waste with an alkali such as dilute sodium hydroxide at an elevated temperature so as to dissolve proteins present in the shell waste. Prolonged alkaline treatment of crustacean shell waste under severe conditions is known to cause, for example, depolymerization and deacetylation of the chitin².

Demineralization typically comprises treatment with an acid such as dilute hydrochloric acid so as to dissolve calcium carbonate present in the crustacean shell waste. The conventional demineralization process of crustacean shell waste may be costly and cause environmental problems. The use of a strong acid such as hydrochloric acid has been shown, for example, to harm the physiochemical properties of chitin³, result in harmful effluent wastewater and/or increase the cost of the chitin purification process.

Acid and alkali treatments alone are known to produce a colored chitin produce. It therefore may be useful for commercial acceptability to decolorize the chitin from crustacean sources by removing pigments or bleaching. Sodium hypochlorite solution is an example of a reagent which has been used in a method for pigment removal from crustacean shell waste⁴.

Deacetylation typically comprises treating the chitin which has been isolated by the deproteinization and demineralization steps with an alkali such as concentrated sodium or potassium hydroxide at elevated temperature so as to convert chitin to chitosan by removal of 60% or more of the acetyl groups. The N-acetyl groups cannot be removed by acidic reagents without hydrolysis of the polysaccharide, thus, alkaline methods must be employed for N-deacetylation⁵.

Therefore, the standard process for producing chitosan is, for example, time consuming, labour intensive, and uses large volumes of water and chemicals which can, for example, result in significant chemical and/or water pollution. Further, the chitosan produced is known to be typically high in heavy metals, of an inconsistent molecular weight, and/or has protein impurities. For example, the chitosan produced from the standard process using a crustacean source can include the same shellfish allergen proteins, which a significant number of the human population is known to be allergic to⁶.

The majority of chitosan produced today is from crustacean sources, which use the extraction procedure outlined above. However, processes for producing chitosan from fungal biomass are known. The cell wall of fungal biomass contains from about 2-20% chitin and/or chitosan⁷. While there are some exceptions, most fungi contain both chitin and chitosan simultaneously.

For example, processes for removing chitin from waste mushrooms such as waste mushrooms from an Agaricus bisporus mushroom farm are known. The chitin obtained is then deacetylated to prepare a final chitosan product with higher purity than that of a crustacean-based chitosan.

U.S. Pat. No. 7,556,946 discloses a method for isolating cell wall derivatives such as chitin from fungal or yeast biomass and the preparation of chitosan from chitin. The isolation of the chitin from the biomass comprises contacting the biomass with a basic solution, contacting the alkali-insoluble fraction obtained from that step with an acidic solution to obtain a suspension of acidified alkali-insoluble fraction and contacting the acidified suspension with β-glucanase enzymes to obtain the cell wall derivatives.

U.S. Pat. No. 7,413,881 discloses a method of obtaining chitosan from microbial biomass, such as fungal biomass. The method comprises providing a consistent chitin-containing biomass, reacting the chitin-containing biomass in a caustic aqueous solution at elevated temperature to convert the chitin to chitosan and separating the chitosan from the caustic solution.

EP Patent Application Publication No. 0 542 249 discloses a method for preparing chitosan comprising deproteinizing cultured filamentous fungi with an alkali at elevated temperature to obtain alkali-insoluble substances and subsequently extracting chitosan from the alkali-insoluble substances with an aqueous solution of an organic acid.

Due to the differences in structure between the fungal biomass and the crustacean shells such as the lack of calcium carbonate in the fungal biomass, the concentration of the acids and bases used may, for example, be less than that used for a process using crustacean waste shells and steps can be eliminated, such as grinding steps and some of the washing and drying steps. Accordingly, producing chitosan from fungal biomass typically uses less chemicals and water than a process using crustacean waste shells as a source. However, known processes using fungal biomass still use significant volumes of water and/or chemicals as well as energy to heat solutions during the process.

Chitosan is used in a wide range of applications. The applications a particular chitosan is useful for may depend, for example, on its quality. Low quality chitosan is used, for example, in high volumes for wastewater treatment and agriculture. High-grade chitosan is used, for example, but not limited to, in medical devices, pharmaceuticals, nanotechnology and cosmetics. In medical and pharmaceutical applications it is useful to use a non-animal derived chitosan.

### SUMMARY

In the present studies, a method for chitosan production is disclosed which comprises allowing the fungal biomass to be exposed to the organic acid produced by that fungal biomass so as to “auto-extract” the acid-soluble chitosan over time into the aqueous medium the fungal biomass was grown in. The chitosan was then precipitated out of the chitosan-enriched aqueous medium, for example, by adding in a base to reach a pH of about 8-9. Chitosan precipitate was then collected, for example, by centrifugation or filtration and dried using a spray dryer or lyophilization. In some embodiments, the collected chitosan was further purified by dissolving in an acidic solution and filtering to remove acid-insoluble impurities. The chitosan in solution was then re-precipitated by adding in a base to reach a pH of about 8-9. The chitosan precipitate was collected by centrifugation or filtration and dried using a spray dryer or lyophilization.

Accordingly, the present disclosure includes a method for chitosan production comprising:
growing a biomass comprising an organic acid-producing, chitosan-containing fungus in an aqueous medium under conditions to obtain a chitosan-reduced biomass and a chitosan-enriched aqueous medium;
separating the chitosan-reduced biomass from the chitosan-enriched aqueous medium; and
separating the chitosan from the chitosan-enriched aqueous medium.

In an embodiment, separating the chitosan from the chitosan-enriched aqueous medium comprises:
adding a base to the chitosan-enriched aqueous medium under conditions to precipitate the chitosan; and
separating the precipitated chitosan from the aqueous medium.

In an embodiment, the conditions to obtain a chitosan-reduced biomass and a chitosan-enriched aqueous medium comprise growing the biomass in the aqueous medium for a first period of time at a first temperature value then raising the temperature to a second temperature value and growing the biomass in the aqueous medium for a second period of time.

In an embodiment, the conditions to precipitate the chitosan comprise adding an aqueous solution of the base to the chitosan-enriched aqueous medium. In an alternative embodiment, the conditions to precipitate the chitosan comprise adding the base in solid form to the chitosan-enriched aqueous medium. In another embodiment, the base comprises sodium hydroxide.

In an embodiment, the precipitated chitosan is separated from the aqueous medium via a physical means of separation.

In an embodiment, the method further comprises:
mixing the separated chitosan with an acidic aqueous solution under conditions to obtain a chitosan-containing aqueous solution and to precipitate acid-insoluble impurities;
separating the acid-insoluble impurities from the chitosan-containing aqueous solution;
adding a base to the chitosan-containing aqueous solution under conditions to re-precipitate the chitosan; and
separating the re-precipitated chitosan from the aqueous solution.

In an embodiment, the acid-insoluble impurities are separated from the chitosan-containing aqueous solution via a physical means of separation. In another embodiment, the re-precipitated chitosan is separated from the aqueous solution via a physical means of separation.

Other features and advantages of the present disclosure will become apparent from the following detailed description. It should be understood, however, that the detailed description and the specific examples while indicating embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The present disclosure will now be described in greater detail with reference to the drawings, in which:

FIG. 1 shows an exemplary scanning electron microscopy (SEM) micrograph of chitosan produced from a mushroom waste source using a standard method. The scale bar corresponds to 50 μm.

FIG. 2 shows an exemplary SEM micrograph of chitosan produced according to an embodiment of the method of the present disclosure. The scale bar corresponds to 50 μm.

FIG. 3 shows an exemplary SEM micrograph of chitosan produced according to an embodiment of the method of the present disclosure at higher magnification. The scale bar corresponds to 2 μm.

FIG. 4 shows an exemplary X-Ray Diffraction (XRD) Analysis of a chitosan standard.

FIG. 5 shows an exemplary XRD of chitosan produced via an embodiment of the method of the present disclosure.

FIG. 6 shows (A) an exemplary ¹³C Cross Polarization Magic Angle Spinning (CP/MAS) NMR (spinning at 7 kHz) of the chitosan collected from an exemplary citric acid production effluent before any further purification. The spectrum shown in (A) has been broken down to show the non-chitosan (B) and chitosan (C) components of the sample analyzed.

### DETAILED DESCRIPTION

### I. Definitions

Unless otherwise indicated, the definitions and embodiments described in this and other sections are intended to be applicable to all embodiments and aspects of the present disclosure herein described for which they are suitable as would be understood by a person skilled in the art.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

The term “suitable” as used herein means that the selection of the particular conditions would depend on the specific manipulation or operation to be performed, but the selection would be well within the skill of a person trained in the art. All methods described herein are to be conducted under conditions sufficient to provide the desired product.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this disclosure, the singular forms “a”, “an” and “the” include plural references unless the content clearly dictates otherwise. For example, an embodiment including “a base” should be understood to present certain aspects with one base or two or more additional bases.

In embodiments comprising an “additional” or “second” component, such as an additional or second base, the second component as used herein is chemically different from the other components or first component. A “third” component is different from the other, first, and second components, and further enumerated or “additional” components are similarly different.

The term “organic acid-producing, chitosan-containing fungus” as used herein refers to a fungus that intracellularly produces one or more organic acids which are exported to the environment in which the fungus is growing and that also contains chitosan. In an embodiment, the chitosan is a naturally-occurring component of the cell wall of the fungi. It will be appreciated by a person skilled in the art that a variety of organic acids such as carboxylic acids or dicarboxylic acids are produced by chitosan-containing fungi through various metabolic pathways and the selection of a particular fungus can be made by a person skilled in the art.

The term “chitin” as used herein refers, for example to a linear polymer comprising N-acetyl-(D)-glucosamine units which have been connected through a β-(1,4) bond and includes random copolymers of N-acetyl-(D)-glucosamine and (D)-glucosamine having less than 60% (D)-glucosamine units.

The term “chitosan” as used herein refers, for example to a random copolymer comprising N-acetyl-(D)-glucosamine and (D)-glucosamine units which have been connected through a τ3-(1,4) bond having 60% or greater (D)-glucosamine units.

The term “fungal biomass” or “biomass” as used herein refers to a mass of fungal material produced via the fermentation.

I. Methods

In the studies of the present disclosure, a method for chitosan production is disclosed which differs from known processes. In the method for chitosan production of the present studies, the fungal biomass was allowed to be exposed to the organic acid produced by the biomass to “auto-extract” the acid-soluble chitosan over time into the aqueous medium the biomass was grown in. The chitosan was then separated from the chitosan-enriched aqueous medium for example, by precipitation by adding in a base to reach a pH of about 8-9. In some embodiments, the chitosan was further dissolved in acidic solution and filtered or centrifuged to remove acid-insoluble impurities. The acidic solution was exposed, for example, to a base to precipitate the chitosan at a pH of about 8-9. It certain embodiments, chitosan precipitate was collected by centrifugation or filtration and dried using a spray dryer or lyophilization. In the method of the present studies, no additional water was used beyond that used to culture the fungi, the amount of alkaline chemicals was significantly reduced in comparison to known processes, very little or no acid was used other than that naturally produced by the fungi being cultured, and heating of the components used in the method was optional.

Accordingly, the present disclosure includes a method for chitosan production comprising:
growing a biomass comprising an organic acid-producing, chitosan-containing fungus in an aqueous medium under conditions to obtain a chitosan-reduced biomass and a chitosan-enriched aqueous medium;
separating the chitosan-reduced biomass from the chitosan-enriched aqueous medium; and
separating the chitosan from the chitosan-enriched aqueous medium.

It will be appreciated by a person skilled in the art that any suitable method of separating the chitosan from the chitosan-enriched aqueous medium will be acceptable and the selection of a suitable method can be made by a person skilled in the art. In an embodiment, separating the chitosan from the chitosan-enriched aqueous medium comprises:
adding a base to the chitosan-enriched aqueous medium under conditions to precipitate the chitosan; and
separating the precipitated chitosan from the aqueous medium.

It will be appreciated by a person skilled in the art that a variety of chitosan-containing fungi produce one or more organic acids and the selection of a suitable organic acid-producing, chitosan-containing fungus for the methods of the present disclosure can be made by a person skilled in the art. In an embodiment, the chitosan-containing, organic acid-producing fungus is from a family selected from Mucoraceae, Trichocomaceae, Nectriaceae, Dipodascaceae and Saccharomycetaceae.

In another embodiment, the organic acid-producing, chitosan-containing fungus is selected from Saccharomyces spp., Rhodotorula spp., Aspergillus spp., Rhizopus spp., Penicillium spp., Paecilomyces spp. and Candida spp.

In another embodiment, the organic acid-producing, chitosan-containing fungus is selected from Saccharomyces cerevisiae, Rhodotorula pichia, Aspergillus niger, Aspergillus itaconicus, Aspergillus oryzae, Aspergillus terreus, Aspergillus falvus, Rhizopus oryzae, Rhizopus nigricans, Penicillium vermiculatum, Penicillium simplicissimum, Paecilomyces variotii and Candida guilliermondii. In a further embodiment, the organic acid-producing, chitosan-containing fungus is selected from Aspergillus niger and Rhizopus oryzae. It is an embodiment that the organic acid-producing, chitosan-containing fungus is Rhizopus oryzae.

It will be appreciated by a person skilled in the art that a variety of organic acids are produced by chitosan-containing fungi. For example, the organic acid produced by the chitosan-containing fungi is a carboxylic acid or a dicarboxylic acid, for example, citric acid, gluconic acid, itaconic (methylenesuccinic) acid, L-lactic acid, oxalic acid, fumaric acid, L-malic acid, succinic acid, (−)-trans-2,3-epoxysuccinic acid, gibberelic acid, meso-tartaric acid, kojic acid (5-hydroxy-2-(hydroxymethyl)-4-pyrone), gallic acid (3,4,5-trihydroxybenzoic acid) or a mixture thereof.

In an embodiment, the organic acid-producing, chitosan-containing fungus produces an organic acid selected from citric acid, gluconic acid, itaconic acid, L-lactic acid, oxalic acid, fumaric acid, L-malic acid, succinic acid, (−)-trans-2,3-epoxysuccinic acid, gibberelic acid, meso-tartaric acid, kojic acid, gallic acid and a mixture thereof. In another embodiment, the organic acid-producing, chitosan-containing fungus produces an organic acid selected from citric acid, gluconic acid, itaconic acid, L-lactic acid, oxalic acid, fumaric acid, L-malic acid, succinic acid, (−)-trans-2,3-epoxysuccinic acid, gibberelic acid, meso-tartaric acid, kojic acid and gallic acid.

In a further embodiment, the organic acid-producing, chitosan-containing fungus produces L-lactic acid, fumaric acid, L-malic acid, citric acid or a mixture thereof. It is an embodiment that the organic acid-producing, chitosan-containing fungus produces L-lactic acid, fumaric acid, L-malic acid or a mixture thereof. In an embodiment, the organic acid-producing, chitosan-containing fungus produces L-lactic acid. In another embodiment, the organic acid-producing, chitosan-containing fungus produces fumaric acid, L-malic acid or a mixture thereof. In a further embodiment, the organic acid-producing, chitosan-containing fungus produces citric acid.

In an embodiment, the conditions to obtain a chitosan-reduced biomass and a chitosan-enriched aqueous medium comprise growing the organic acid-producing, chitosan-containing fungus in an aqueous medium where the organic acid produced by the fungus renders that aqueous medium acidic and thus solubilizes at least a portion of the chitosan in the fungus. Therefore, growth of the fungus in the aqueous medium results in so-called “auto-extracting” of chitosan from the fungus to provide a chitosan-reduced biomass and a chitosan-enriched aqueous medium. In an embodiment, the conditions to obtain a chitosan-reduced biomass and a chitosan-enriched aqueous medium further comprise growing the fungus under conditions, such as temperature, pressure, agitation, time and nutrients in the aqueous medium, to optimize the growth of the fungus, production of organic acid and/or extraction of chitosan into the aqueous medium.

It will be appreciated by a person skilled in the art that the conditions to optimize the growth of the fungus, production of organic acid and/or extraction of chitosan into the aqueous medium, will vary depending on, for example, the identity of the fungus, the time available and/or the purity of chitosan desired, but none-the-less, can be determined using methods known in the art. It will be appreciated by a person skilled in the art that aqueous media for growing fungi typically contain a carbon source, for example, glucose, dextrose, fructose, mannose, sucrose or a mixture thereof and a nitrogen source, for example, hydrolyzed soymeal, yeast extract, peptone such as soy peptone, protein, amino acids, ammonium salts such as ammonium sulfate, nitrate salts or a mixture thereof. Aqueous media for growing fungi which are known to be naturally deficient in a vitamin will also typically contain the vitamin. Aqueous media for growing fungi also can contain plant growth hormones such as gibberelic acid. The selection of a suitable aqueous medium for growing a particular fungus can be made by a person skilled in the art.

In an embodiment, the conditions to obtain a chitosan-reduced biomass and a chitosan-enriched aqueous medium comprise growing the biomass in the aqueous medium for a time of about 3 hours to about 5 days, about 4 hours to about 48 hours, about 8 hours to about 36 hours or about 20 hours to about 28 hours at a temperature of about 10° C. to about 60° C., about 15° C. to about 50° C., about 20° C. to about 40° C. or about 26° C. to about 32° C.

In another embodiment, the conditions to obtain a chitosan-reduced biomass and a chitosan-enriched aqueous medium comprise growing the biomass in the aqueous medium for a first period of time at a first temperature value then raising the temperature to a second temperature value and growing the biomass in the aqueous medium for a second period of time. It will be appreciated by a person skilled in the art that the second period of time will generally be shorter than the first period of time as while the higher temperature is useful to facilitate extraction of the chitosan such temperatures are generally less desirable for growth of the biomass. The optimum timing and temperatures for a particular biomass can be determined by a person skilled in the art. In an embodiment, the biomass is grown for about 8 hours to about 5 days or about 20 hours to about 28 hours at a temperature of about 20° C. to about 40° C. or about 26° C. to about 32° C. then the temperature is raised to a value of about 50° C. to about 70° C. or about 55° C. to about 65° C. and the biomass is grown for a further about 30 minutes to about 2 hours or about 45 minutes to about 75 minutes.

In an embodiment, the pH of the aqueous medium reaches a value of less than about 6.5, about 2.5 to about 6.5 or about 3 to about 4. In another embodiment, the pH of the aqueous medium reduces until it is substantially maintained at a value of from about 2 to about 4 or about 3.1.

In an embodiment, the biomass is grown in a vessel which is agitated. In another embodiment, the vessel is agitated at a speed of about 50 rpm to about 500 rpm or about 150 rpm to about 400 rpm.

In an embodiment, the biomass is grown at a concentration of about 0.1 g/L to about 1000 g/L or about 1 g/L to about 40 g/L based on the total volume of the biomass and aqueous medium.

In an embodiment, the chitin-reduced biomass is separated from the chitosan-enriched aqueous medium via physical or chemical means, for example by filtration, centrifugation or any other physical or chemical means suitable to remove a liquid fraction from a precipitate. In another embodiment, the chitin-reduced biomass is separated from the chitosan-enriched aqueous medium via a physical means of separation such as via filtration or centrifugation.

In an embodiment, the conditions to precipitate the chitosan comprise adding the base in solid form such as powder or pellets to the chitosan-enriched aqueous medium. In another embodiment, the conditions to precipitate the chitosan comprise adding an aqueous solution of the base to the chitosan-enriched aqueous medium. The selection of a particular form of base to be added to the chitosan-enriched aqueous medium will depend, for example on the base to be added and can be made by a person skilled in the art.

In an embodiment, the base is added to the chitosan-enriched medium until a pH of about 6.5 to about 10 or about 8 to about 9, is obtained. In an embodiment, the base comprises an alkali metal hydroxide, an alkali metal carbonate or an organic base such as ammonia. In another embodiment, the base comprises an alkali metal hydroxide. In a further embodiment, the base comprises sodium hydroxide. It is an embodiment that the concentration of the aqueous solution comprising the base is from about 0.1 M to about 10 M, although any suitable concentration of base will be acceptable.

In an embodiment, the precipitated chitosan is separated from the aqueous medium via filtration, centrifugation, chromatography or any other physical or chemical means suitable to remove a liquid fraction from a precipitate. In another embodiment, the precipitated chitosan is separated from the aqueous medium via filtration, centrifugation or chromatography. In a further embodiment, the precipitated chitosan is separated from the aqueous medium via filtration or centrifugation. It is an embodiment that the precipitated chitosan is separated from the aqueous medium via centrifugation.

In an embodiment, the method of the disclosure further comprises:
mixing the separated chitosan with an acidic aqueous solution under conditions to obtain a chitosan-containing aqueous solution and to precipitate acid-insoluble impurities;
separating the acid-insoluble impurities from the chitosan-containing aqueous solution;
adding a base to the chitosan-containing aqueous solution under conditions to re-precipitate the chitosan; and
separating the re-precipitated chitosan from the aqueous solution.

In an embodiment, the acidic aqueous solution comprises an organic acid, an inorganic acid or a combination thereof. In another embodiment, the acidic aqueous solution comprises an organic acid such as lactic acid, acetic acid or combinations thereof. In a further embodiment, the acidic aqueous solution comprises an inorganic acid such as HCl or a similar inorganic acid. In an embodiment, the acidic aqueous solution has a pH of less than about 4, or from about 3 to about 4. In another embodiment of the present disclosure, the concentration of the acid in the acidic aqueous solution is about 5 wt % although any suitable concentration of acid will be acceptable, and depends, for example on the identity of the acid.

In an embodiment, the acid-insoluble impurities are separated from the chitosan-containing aqueous solution via filtration, centrifugation or any other physical or chemical means suitable to remove a liquid fraction from a precipitate. In another embodiment, the acid-insoluble impurities are separated from the chitosan-containing aqueous solution via a physical means of separation. In a further embodiment, the acid-insoluble impurities are separated from the chitosan-containing aqueous solution by filtration or centrifugation. It is an embodiment that the acid-insoluble impurities are separated from the chitosan-containing aqueous solution via centrifugation.

In an embodiment, the conditions to re-precipitate the chitosan comprise adding the base in solid form such as powder or pellets to the chitosan-containing aqueous solution. In another embodiment, the conditions to precipitate the chitosan comprise adding an aqueous solution of the base to the chitosan-containing aqueous solution. The selection of a particular form of base to be added to the chitosan-containing aqueous solution will depend, for example on the base to be added and can be made by a person skilled in the art.

In an embodiment, the base is added to the chitosan-containing aqueous solution until a pH of about 6.5 to about 10 or about 8 to about 9, is obtained. In an embodiment, the base comprises an alkali metal hydroxide, an alkali metal carbonate or an organic base such as ammonia. In another embodiment, the base comprises an alkali metal hydroxide. In a further embodiment, the base comprises sodium hydroxide. It is an embodiment that the concentration of the aqueous solution comprising the base is from about 0.1 M to about 10 M, although any suitable concentration of base will be acceptable.

In an embodiment, the re-precipitated chitosan is separated from the aqueous solution via filtration, centrifugation, chromatography or any other physical or chemical means suitable to remove a liquid fraction from a precipitate. In another embodiment, the re-precipitated chitosan is separated from the aqueous solution via filtration, centrifugation or chromatography. In a further embodiment, the re-precipitated chitosan is separated from the aqueous solution via filtration or centrifugation. It is an embodiment that the re-precipitated chitosan is separated from the aqueous solution via centrifugation.

In an embodiment, the method further comprises drying the separated chitosan. In another embodiment, the chitosan is dried by a method comprising spray drying or lyophilization.

In an embodiment, the method is operated continuously.

The following non-limiting examples are illustrative of the present application:

### EXAMPLES

### Example 1

### Production of Chitosan from Rhizopus oryzae

I. Materials and Methods

The Rhizopus oryzae fungal biomass was allowed to grow in media comprising the lactic acid produced by the fungal biomass in a fermenter so as to “auto-extract” the chitosan over time. The chitosan was then precipitated out of the media by adding a solid base (NaOH) to reach a pH of about 8-9. Chitosan precipitate was then collected by centrifugation, and dried using a spray dryer or lyophilization.

In some experiments, the precipitated chitosan was dissolved in an acidic solution and centrifuged to remove acid-insoluble impurities. The acidic solution comprised an organic acid such as lactic acid or acetic acid at a pH low enough to dissolve the chitosan; i.e. around pH 3-4. The further purified chitosan was then re-precipitated from the acidic solution by adding a solid base (NaOH) to reach a pH of about 8-9 and the re-precipitated chitosan was then collected by centrifugation. The collected re-precipitated chitosan was then dried using a spray dryer or lyophilization.

II. Results

Tables 1-3 show the results for studies examining how much chitosan the “auto-extraction” method of the present disclosure can obtain, as a function of how much biomass is present under three different conditions.

Table 4 shows the results for the change in pH of the growth media over time as the Rhizopus oryzae produces lactic acid and it accumulates in the fermenter tank. The growth media used contained a carbon source, a nitrogen source and a plant growth hormone. The Rhizopus oryzae was inoculated at time=0. Chitosan was observed to be soluble under a pH of about 6.5.

Table 5 shows the results of addition of NaOH pellets to precipitate chitosan. 10 L of media was taken after each 40 L fermenter growth period after 24 hours and the amount of base needed was recorded. It was found that for every liter of growth media it is useful to add 0.684 grams of NaOH to precipitate the chitosan.

III. Discussion

In the methods of the present disclosure, fungal biomass is grown using a submerged fermentation method. Such a method can, for example be non-reliant on waste biomass like known methods of chitosan production which use crustacean waste shells or mushroom waste. The present method also uses significantly less chemicals, water and/or energy than known processes.

The fungal strain Rhizopus oryzae typically contains about 1-10% chitosan in the total biomass. The chitosan in the present studies is water soluble, but only at a pH<6.5, which means that it is soluble only in acidic solutions. Rhizopus oryzae produces lactic acid. Therefore while R. oryzae is growing in the liquid media, it is producing lactic acid, which is released into the liquid media. This solubilizes the chitosan in the cell wall of the fungus, and extracts it out into the liquid media over time.

After inoculation, as the Rhizopus oryzae begins to produce lactic acid it lowers the pH of the liquid media and it begins to extract its own chitosan simultaneously. The pH of the liquid media has been observed to be lowered to a pH of about 3 or less after a 24 hour period. However, the growth and metabolic function of R. oryzae is inhibited below a pH of about 4.5. The acidic solution containing the dissolved chitosan was then collected, the biomass filtered out and discarded, and a base (the present studies used sodium hydroxide in pellet form) added to precipitate the chitosan at a pH of about 7-10. The chitosan precipitate was then collected from the liquid fraction by centrifugation and the liquid supernatant discarded. In some trials, the isolated chitosan was then dried. In other trials, the isolated chitosan was then dissolved in an acidic solution and filtered to remove acid-insoluble material. The chitosan solution was exposed to a base to adjust the pH to about 8-9 and precipitate the further purified chitosan. The chitosan precipitate was then collected from the liquid fraction by centrifugation and the liquid supernatant discarded. The chitosan was then dried.

The amount of chitosan extracted was observed to depend on the following factors: The volume of liquid media, which impacts how long it will take to get the pH to an acidic pH; the amount of Rhizopus oryzae biomass produced, which depends, for example on the amount of nutrients in the media and/or the growth time; and the temperature in the fermenter tank, as increasing the temperature was observed to result in a more efficient extraction of chitosan from the cell wall. These factors will now be discussed in greater detail.

The Rhizopus oryzae used in the present studies was grown using submerged fermentation, in a tank filled with water and growth media to feed it. As the fungus produced increasing biomass, increasingly lactic acid was produced and accumulated in the liquid media. The liquid media started out at a pH of about 6.5, and decreased steadily to a pH of about 2-3 as more lactic acid accumulated in the media.

Generally, the more Rhizopus oryzae biomass produced, the more chitosan will be produced and higher amounts of chitosan will be extracted into the growth media. Biomass production depends, for example on the availability of nutrients in the growth media, the temperature of the fermenter, the aeration, the pH of the growth media (Rhizopus oryzae is acid tolerant but only to an extent) and/or the volume of the spore inoculate.

An optimal growth temperature of Rhizopus oryzae is about 30° C. At increasingly higher temperatures growth slows and eventually stops. However, at higher temperatures, for example temperatures above about 50° C. the acid more readily solubilizes the chitosan from the biomass cell walls and therefore more chitosan is extracted into the liquid media.

IV. Microscopy

Scanning Electron Microscopy (SEM) was used to obtain exemplary micrographs of the chitosan produced from the methods of the present studies. A comparison was made to SEM micrographs of chitosan produced from a mushroom waste source using a standard process.

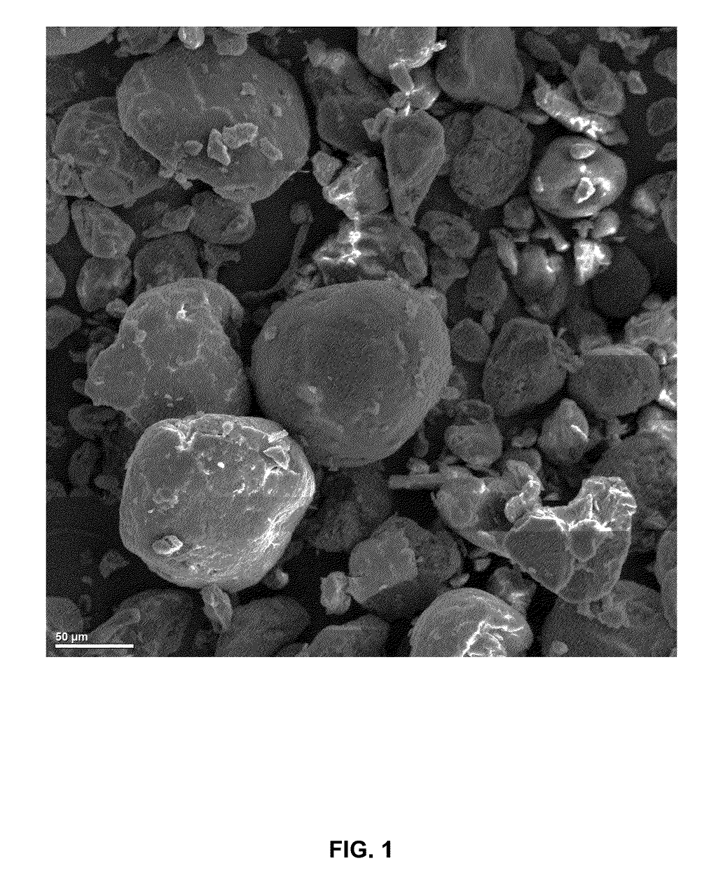

The chitosan in FIGS. 1-3 was dissolved in a solution of lactic acid at a concentration of 8 g/L, concentrated by 30% then precipitated into methanol. This resulted in a milky white suspension that did not dissolve overnight, suggesting micron-sized particles.

As can be seen in FIG. 1, the chitosan produced from a mushroom waste source using a standard process is generally smooth and granular with a particle size of 10-100 microns.

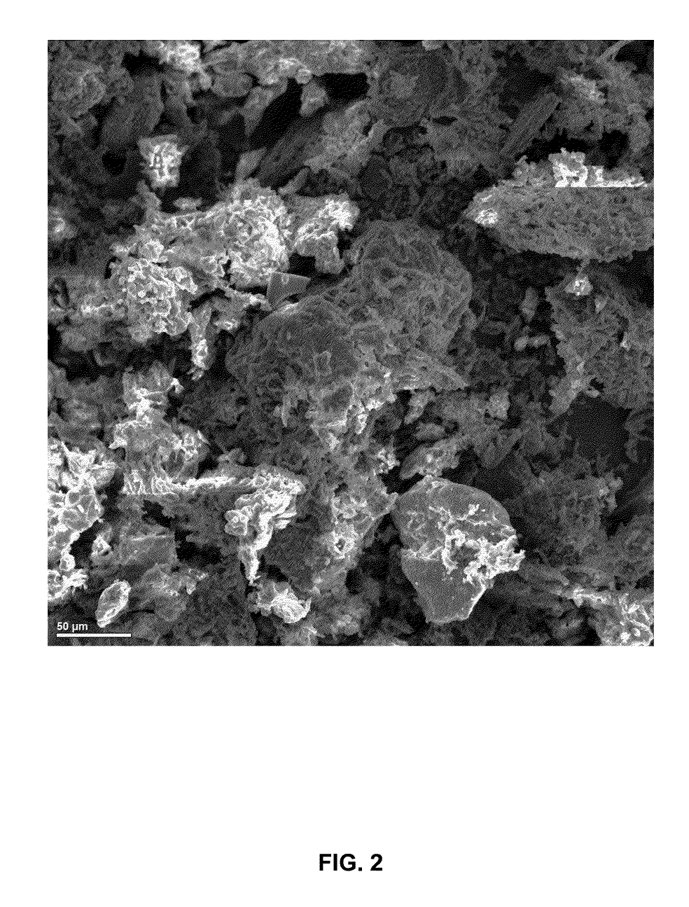

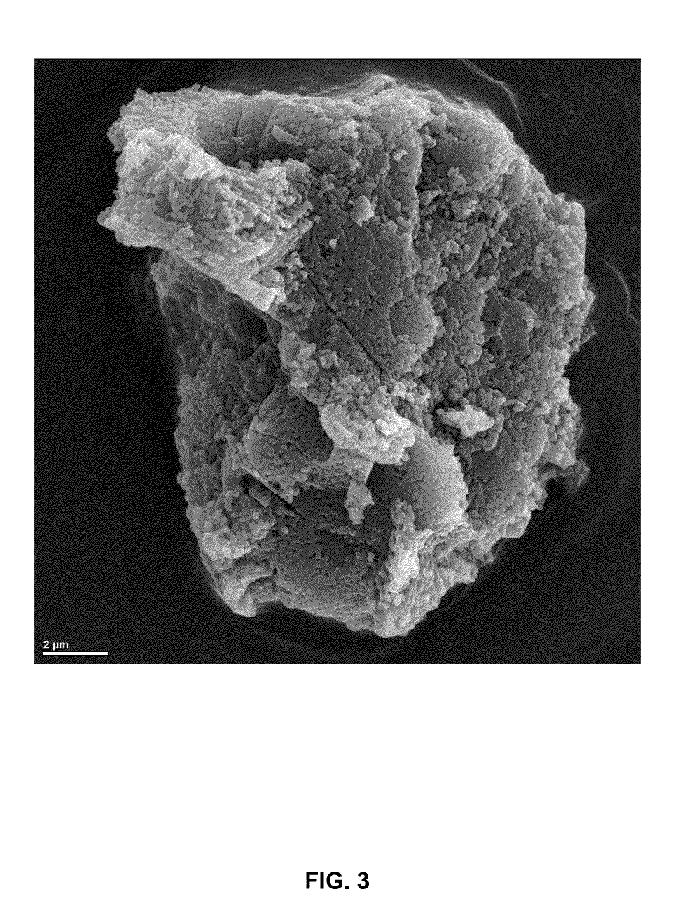

In contrast, as can be seen in FIGS. 2 and 3, chitosan produced from the methods of the present studies has a morphology which is more flakey and porous. This morphology is desirable and represents a “natural” chitosan that has not been subjected to harsh chemical treatments of standard processes. A more porous chitosan is useful, for example for applications in wound care and drug delivery.

FIG. 2 shows chitosan produced using Rhizopus oryzae grown in liquid media at 30° C. for 48 hours. The chitosan was precipitated using pellet form sodium hydroxide and was collected by centrifugation. It was lyophilized to remove any moisture. Chitosan was removed from the fungal biomass as the lactic acid produced by the fungus reduced the pH of the liquid media to an acidic pH. It is a granular powder with a particle size of 10-150 microns and appears to be composed of flake-like particles, randomly oriented.

FIG. 3 shows the chitosan of FIG. 2 at a higher magnification. As can be seen in the SEM micrograph of FIG. 3, agglomerates of 10-15 microns were obtained that were composed of smaller particles and/or flakes having a diameter between 200-500 nanometers.

V. X-Ray Diffraction (XRD)

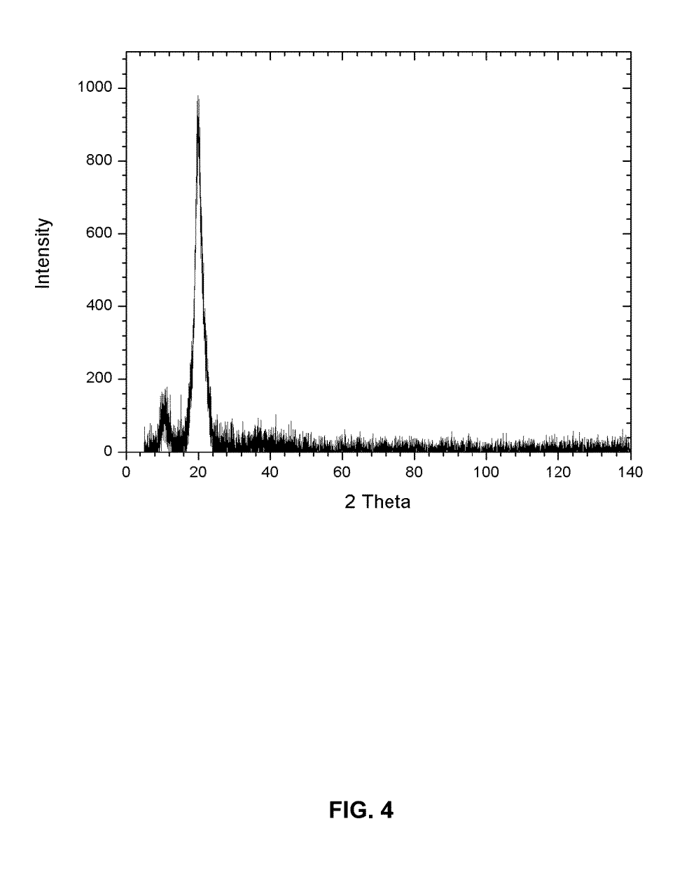

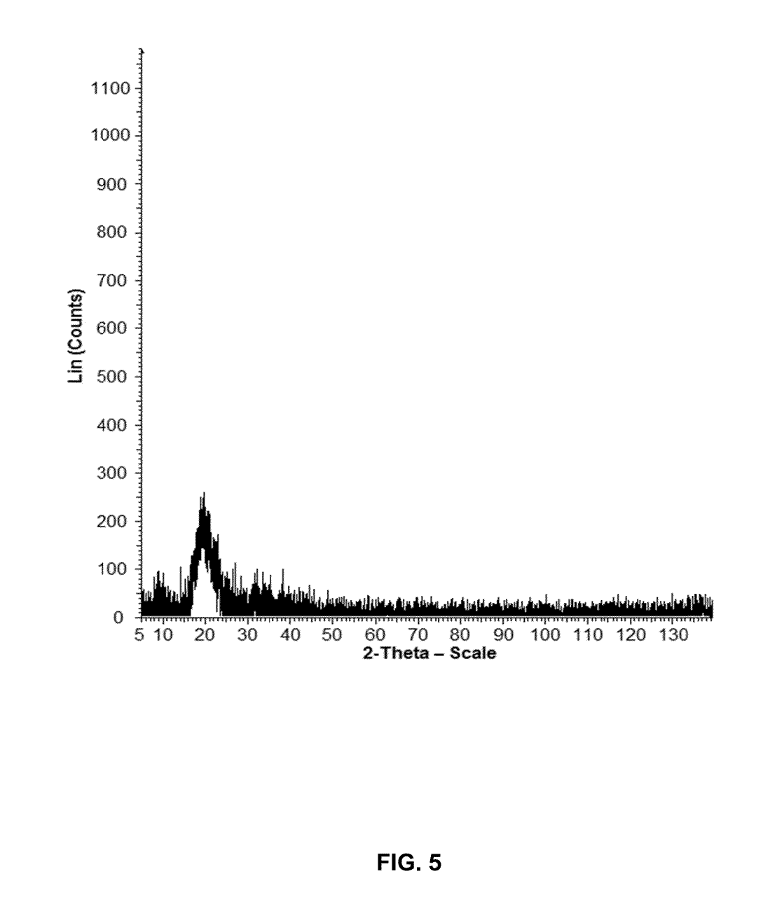

The XRD analysis was performed at 25° C. using a Broker AXS D8 Advance solid-state powder diffraction XRD system. FIG. 4 shows an exemplary XRD diffractogram of a chitosan standard. The chitosan standard was obtained from Sigma Aldrich. FIG. 5 shows an exemplary XRD powder diffractogram of a sample of chitosan produced via the method of the present studies. As can be seen from these figures the broad diffraction peaks at 2 8=10° and 20° are visible for the sample of chitosan produced via the method of the present studies and consistent with the chitosan standard.

### Example 2

### Production of Chitosan from Aspergillus niger

Commercial production of organic acids such as citric acid may comprise growing an acid-producing fungus in submerged fermentation. For example, citric acid is produced by the fungal species Aspergillus niger using such a method. A. niger also happens to have significant amounts of chitosan in its cell wall. The results of the present studies suggest that chitosan could therefore be obtained once the citric acid is isolated from the liquid media.

Such methods of citric acid production typically will remove the citric acid and leave the liquid media to be discarded. The present studies have tested the waste liquid from a commercial citric acid producer and found chitosan to be present at concentrations of from 0.02 g/L to 0.12 g/L.

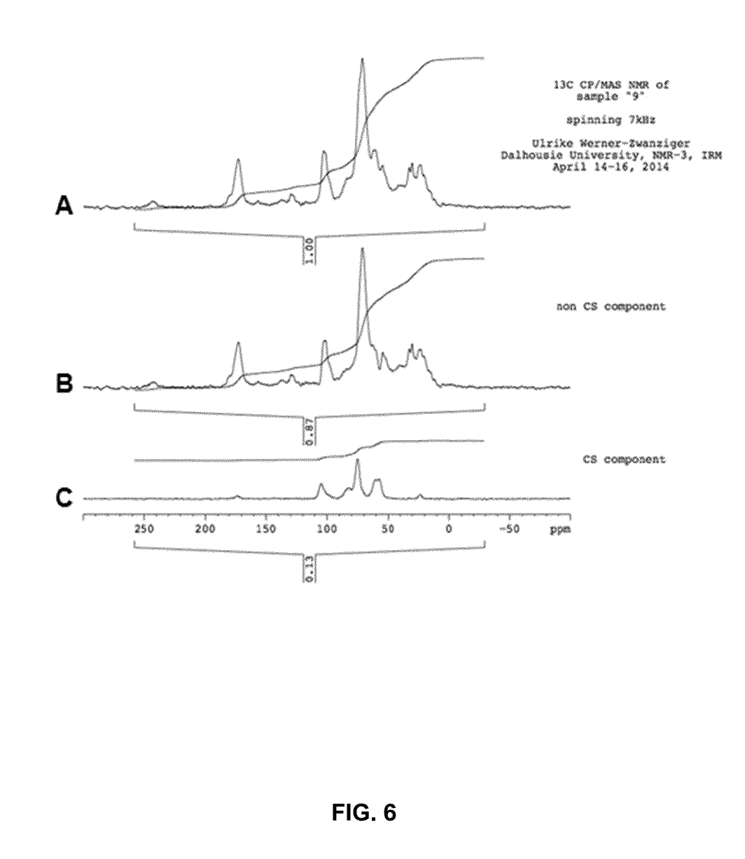

FIG. 6 shows an exemplary ¹³C Cross Polarization Magic Angle Spinning (CP/MAS) NMR (spinning 7 at kHz) of the chitosan collected from citric acid production effluent before any purification. The spectrum shown in (A) has been broken down to show the non-chitosan (B) and chitosan (C) components of the sample analyzed. These results show that this effluent from citric acid production contains chitosan.

To produce chitosan from the waste liquid of citric acid production, the pH is increased using a base. In the present studies, sodium hydroxide (NaOH) was used to increase the pH of the waste liquid to a pH of about 7-10 and this caused the chitosan to precipitate out of the solution. From there the chitosan precipitate is filtered out and dried, or alternatively the whole mixture is centrifuged to remove the liquid fraction and then dry the collected chitosan. The chitosan is purified by dissolving it in an acidic solution and filtering or centrifuging to isolate acid-insoluble material. The chitosan solution is then precipitated with NaOH around pH 7-10, collected and dried.

While the present disclosure has been described with reference to what are presently considered to be the preferred examples, it is to be understood that the disclosure is not limited to the disclosed examples. To the contrary, the present disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### FULL CITATIONS FOR DOCUMENTS REFERRED TO IN THE SPECIFICATION

¹Aranaz, I., M. Mengibar, et al. “Functional Characterization of Chitin and Chitosan,” Current Chemical Biology, vol. 3, No. 2, pp. 203-230, May 2009.
²Mao, et al. “The depolymerization of chitosan: effects on physicochemical and biological properties,” International Journal of Pharmaceutics, vol. 281, pp. 45-54, Aug. 20, 2004.
³Kato, et al. “Depolymerization of N-succinyl-chitosan by hydrochloric acid,” Carbohydrate Research, vol. 337, No. 6, pp 561-564, Mar. 15, 2002.
⁴Moorjani, M. N., Achutha, V., and Khasim, D. I. “Parameters affecting the viscosity of chitosan from prawn waste,” J. Food Sci. Technol,. vol. 12. pp. 187-189, 1975.

⁵Muzzarelli, R. A. A. “Enzymatic synthesis of chitin and chitosan. Occurrence of chitin”. In: Chitin (Muzzarelli, R. A. A., ed.), pp. 5-44. Pergamon Press, New York, N.Y. 1977.

⁶Anas, et al. Characterization of Seafood Proteins Causing Allergic Diseases, “Allergic Diseases—Highlights in the Clinic, Mechanisms and Treatment”, Celso Pereira (Ed.), ISBN: 978-953-51-0227-4, InTech, DOI: 10.5772/25316. 2012.
⁷B. Aguilar-Uscanga, J. M. Francois, “A study of the yeast cell wall composition and structure in response to growth conditions and mode of cultivation,” Letters in Applied Microbiology, vol. 37, pp. 268-274, Jun. 19, 2003.

TABLE 1

| | | | | Total | Chitosan |
|---|---|---|---|---|---|
| | | | End pH | chitosan | (g) per |
| Test | Biomass | Chitosan | of | (g) from | 1 g of |
| No. | (g) | (g/L) | media | 35 L Media | biomass |
| 1 | 164.5 | 0.11 | 3.12 | 3.85 | 0.0234 |
| 2 | 189.42 | 0.134 | 3.22 | 4.69 | 0.0248 |
| 3 | 169.24 | 0.13 | 3.28 | 4.55 | 0.0194 |
| 4 | 177.8 | 0.12 | 3.13 | 4.2 | 0.0179 |
| Average: | 175.24 | 0.1235 | 3.19 | 4.3225 | 0.021375 |

Conditions: Aqueous media included a carbon source, a nitrogen source and a plant growth hormone; Temperature 30° C.; Duration 24 hours; Agitation 340 rpm; Aeration 2.1 vvm.

TABLE 2

| | | | | Total | Chitosan |
|---|---|---|---|---|---|
| | | | End pH | chitosan | (g) per |
| Test | Biomass | Chitosan | of | (g) from | 1 g of |
| No. | (g) | (g/L) | media | 35 L Media | biomass |
| 1 | 158.3 | 0.142 | 3.2 | 4.97 | 0.0314 |
| 2 | 184.13 | 0.16 | 3.11 | 5.6 | 0.0304 |
| 3 | 150.23 | 0.114 | 3.12 | 3.99 | 0.0266 |
| 4 | 174.3 | 0.162 | 3.1 | 5.67 | 0.0325 |
| Average: | 166.74 | 0.1445 | 3.13 | 5.0575 | 0.030225 |

Conditions: Aqueous media included a carbon source, a nitrogen source and a plant growth hormone; Temperature 37° C. until hour 23 at which point the temperature was increased to 60° C. for 1 hour; Duration 24 hours; Agitation 340 rpm; Aeration 2.1 vvm.

TABLE 3

| | | | | Total | Chitosan |
|---|---|---|---|---|---|
| | | | End pH | chitosan | (g) per |
| Test | Biomass | Chitosan | of | (g) from | 1 g of |
| No. | (g) | (g/L) | media | 35 L Media | biomass |
| 1 | 279.3 | 0.215 | 3.2 | 7.525 | 0.0269 |
| 2 | 255.2 | 0.223 | 3.37 | 7.81 | 0.0306 |
| 3 | 280.1 | 0.192 | 3.1 | 6.72 | 0.0239 |
| 4 | 283.2 | 0.241 | 3.3 | 8.435 | 0.0298 |
| Average: | 274.45 | 0.21775 | 3.2425 | 7.6225 | 0.0278 |

Conditions: Aqueous media included twice as much carbon source than was used in the studies of Table 1, a nitrogen source and a plant growth hormone; Temperature 37° C.; Duration 24 hours; Agitation 340 rpm; Aeration 2.1 vvm.

TABLE 4

| Hour | pH |
|---|---|
| 0 | 6.5 |
| 2 | 6.5 |
| 4 | 6.5 |
| 6 | 6.5 |
| 8 | 6.5 |
| 10 | 6.4 |
| 12 | 6.3 |
| 14 | 6.0 |
| 16 | 5.5 |
| 18 | 4.4 |
| 20 | 3.6 |
| 22 | 3.2 |
| 24 | 3.1 |

Conditions: Aqueous media included a carbon source, a nitrogen source and a plant growth hormone; Temperature 30° C.; Duration 24 hours; Agitation 340 rpm; Aeration 2.1 vvm.

TABLE 5

| | pH of | NaOH | End | Precip- |
|---|---|---|---|---|
| Run | growth media | added (g) | pH | itation |
| 1 | 3.2 | 6.85 | 9 | yes |
| 2 | 3.3 | 7 | 9 | yes |
| 3 | 3.1 | 6.5 | 9 | yes |
| 4 | 3.1 | 7 | 9 | yes |
| Average: | 3.18 | 6.8375 | 9 | yes |

1. A method for chitosan production comprising:
growing a biomass comprising an organic acid-producing, chitosan-containing fungus in an aqueous medium under conditions to obtain a chitosan-reduced biomass and a chitosan-enriched aqueous medium;
separating the chitosan-reduced biomass from the chitosan-enriched aqueous medium; and
separating the chitosan from the chitosan-enriched aqueous medium.

2. The method of claim 1, wherein separating the chitosan from the chitosan-enriched aqueous medium comprises:
adding a base to the chitosan-enriched aqueous medium under conditions to precipitate the chitosan; and
separating the precipitated chitosan from the aqueous medium.

3. The method of claim 1, wherein the conditions to obtain a chitosan-reduced biomass and a chitosan-enriched aqueous medium comprise growing the biomass in the aqueous medium for a time of about 20 hours to about 28 hours at a temperature of about 26° C. to about 32° C.

4. The method of claim 1, wherein the conditions to obtain a chitosan-reduced biomass and a chitosan-enriched aqueous medium comprise growing the biomass in the aqueous medium for a first period of time at a first temperature value then raising the temperature to a second temperature value and growing the biomass in the aqueous medium for a second period of time.

5. The method of claim 2, wherein the conditions to precipitate the chitosan comprise adding the base in solid form to the chitosan-enriched aqueous medium.

6. The method of claim 5, wherein the base comprises sodium hydroxide.

7. The method of claim 2, wherein the conditions to precipitate the chitosan comprise adding an aqueous solution of the base to the chitosan-enriched aqueous medium.

8. The method of claim 7, wherein the base comprises sodium hydroxide.

9. The method of claim 2, wherein the precipitated chitosan is separated from the aqueous medium via a physical means of separation.

10. The method of claim 1, wherein the method further comprises drying the separated chitosan.

11. The method of claim 1, wherein the method further comprises:
mixing the separated chitosan with an acidic aqueous solution under conditions to obtain a chitosan-containing aqueous solution and to precipitate acid-insoluble impurities;
separating the acid-insoluble impurities from the chitosan-containing aqueous solution;
adding a base to the chitosan-containing aqueous solution under conditions to re-precipitate the chitosan; and
separating the re-precipitated chitosan from the aqueous solution.

12. The method of claim 11, wherein the acid-insoluble impurities are separated from the chitosan-containing aqueous solution via a physical means of separation.

13. The method of claim 11, wherein the re-precipitated chitosan is separated from the aqueous solution via a physical means of separation.
